(19) United States
(12) Patent Application Publication
(10) Pub. No.: US 2014/0052015 A1
(43) Pub. Date: Feb. 20, 2014

(54) QEEG Correlates of Effective Cognitive Functioning (memory and problem solving) in Diverse Clinical Conditions

(71) Applicant: Kirtley Thornton, Metuchen, NJ (US)

(72) Inventor: Kirtley Thornton, Metuchen, NJ (US)

(21) Appl. No.: 13/589,986

(22) Filed: Aug. 20, 2012

(51) Int. Cl. A61B 5/0476

(52) U.S. Cl. CPC A61B 5/0476
USPC 600/544

(57) ABSTRACT

A previous patent (Thornton, 2001) by the inventor focused on the quantitative EEG (QEEG) correlates of successful cognition in normal subjects across a variety of cognitive measures. The present patent focuses on three critical cognitive skills (auditory memory, reading memory and problem solving) and examines a database which is comprised of normal subjects as well as subjects with head injuries, attention deficit disorders, learning disabilities, reading disabilities, math disabilities and unspecified clinical/learning problems.

The QEEG correlates of success and failure were examined during the initial input stage of the task (i.e. listening to stories on a CD, reading text silently) and the immediate silent, eyes closed task of recall of the material. The problem solving task (Raven's matrices) QEEG correlates were examined during the administration of the task. The data was separated between children (ages 10-14) and adults (age greater than 14 years).

### BACKGROUND/SPECIFICATIONS

The search for the specific quantitative EEG correlates of specific cognitive abilities under cognitive activation conditions (in normal subjects) has been the subject of much of the inventor's research publication history. However, only a few of these publications (Thornton, 2000, 2001, 2002a, 2002b, 2006, 2009) have presented the actual QEEG correlates of successful and unsuccessful cognition and have focused on the auditory and reading memory tasks. There is no other published research which has examined the relation between the specific QEEG variables and cognition under cognitive activation tasks. Research by others (Thatcher, 2002) have focused on the relations between the QEEG variables obtained during eyes closed, resting conditions and academic/performance measures obtained at a different point in time.

Thornton & Carmody (2009) reported on the problems of using eyes closed data in understanding the actual relations between the QEEG and cognition under cognitive task conditions. The study indicated that the relative power values of theta activity in the eyes closed condition correlated positively with subsequent auditory recall. However, the relative power of theta values are 1) generally considered a negative indicator of cognitive ability and 2) did not correlate with successful performance during the actual task. The article further elaborated on many of the inconsistencies between eyes closed and simple attention tasks (visual, auditory) predictor QEEG variables and subsequent cognitive task QEEG correlates to auditory memory and reading memory. The article concluded that eyes closed data and even simple visual and auditory attention tasks are not useful in understanding or meaningfully predicting what occurs under task conditions. In addition, almost all currently employed databases do not extend the frequency range up to 64 Hertz. The higher “gamma” (32-64 Hz) frequency range is employed in the data presented.

Table 1 presents the information on the sample size for the adult sample. Table 2 presents the information on the children's database.

TABLE 1
Adult

| | Listening | Reading | Problem |
|---|---|---|---|
| | Condition | Condition | Solving |
| Avg. | 39.25 | 38.8 | 37.96 |
| Age | | | |
| Educ. | 13.6 | 13.55 | 13.4 |
| Male | 65 | 61 | 348 |
| Female | 71 | 59 | 351 |
| Total | 136 | 120 | *699 |

*= indicates total number of Ravens matrices tasks administered

Table 2 presents the identification information for the children's sample.

TABLE 2
Children

| | Listening | Reading | Problem |
|---|---|---|---|
| | Condition | Condition | Solving |
| Avg. | 11.08 | 11.03 | 11.8 |
| Age | | | |
| Educ. | 5.62 | 5.57 | 5.94 |
| Male | 26 | 25 | 166 |
| Female | 17 | 17 | 96 |
| Total | 43 | 42 | *262 |

*= indicates total number of Ravens matrices tasks administered

The Thornton (2001) patent had a subject pool of 83 normal adults and 30 children under the age of 13. The present subject pool is approximately 136 adults and 43 children. The 2001 patent only employed normal subjects in the analysis. This sample included all available subjects, including those with brain injuries, learning problems, attention deficit disorder and other clinical conditions not necessarily categorized with these labels (i.e. radiation treatment for cancer). Thus, for adults the sample size represented a 64% increase in size. For the children's group the increase to 43 represents a 43% increase in sample size.

Apart from the issue of the increase in sample size is the issue of greater generalizability of the present results and increase in face validity. The generalizability argument is based upon the inclusion of the diverse set of clinical conditions. Thus, a variable which may not be relevant in a normative sample may be critical in a learning disabled sample. As a metaphor example, a car's performance will be dependent upon a number of engine characteristics and different engine designs will employ different engineering approaches to maximize performance. A normative sample assumes that the general “wiring” is in place and searches for specifics of the “wiring” which improves performance. However, if the alternator isn't working the car won't move. Similarly, in the case of a learning disabled student one or more “wiring” problems which are not present in a normal population may be the cause of the cognitive problems. Thus, it is important to understand the totality of possible responses.

The patent addresses three specific cognitive skills 1) Auditory Memory; 2) Reading Memory; 3) Problem Solving, across the two age ranges studied (children versus adults). The cut off age for the two groups is age 14. Thus, if the subject was over the age of 14 he/she was placed in the adult sample and if under the age of 14, he/she was placed in the children's sample. For each of the memory tasks, the data is analyzed according to whether it was recorded during the input part (while listening or reading) and immediate recall part. The immediate recall tasks involves the subject quietly recalling the information while their eyes are closed. For the Raven's task only the data obtained during the administration of the task is analyzed.

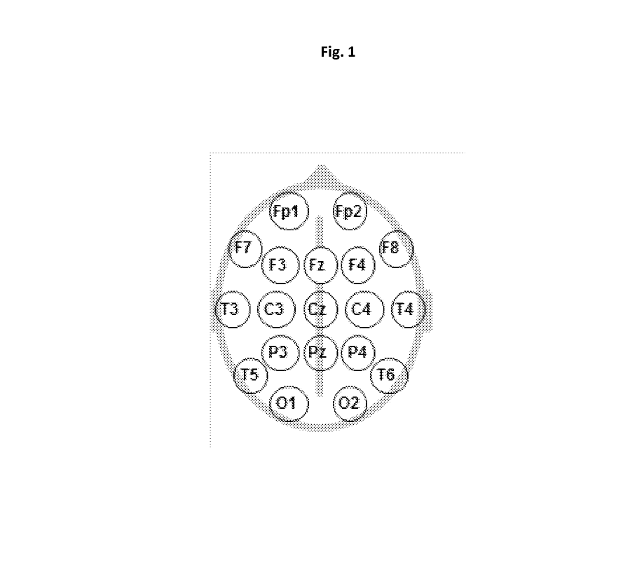

The respective data is analyzed according to whether the variables are positively or negatively correlated with the performance variable. The alpha level was set to 0.10 to allow for maximum inclusion of potentially important variables. A flashlight metaphor is employed in the presentation of the results. The flashlight metaphor states that an individual location is sending out a “beam” to all other 18 locations, within a given frequency. The mini-flashlight metaphor is also employed in the presentations. The mini-flashlight “beam” reports only those connections from a specific location which achieve significance. Thus, while a normal flashlight beam will involve connections from one location to 18 other locations, a mini-flashlight beam may only involve 2 to 5 connections. FIG. 1 presents the 10-20 standard EEG nomenclature for the brain locations. The concept of coherence employs the Lexicor's algorithm of Spectral Correlation Coefficients and Lexicor's Phase algorithm. Different hardware/software manufacturers employ different algorithms to calculate these values.

Activation Measures

Absolute Magnitude: the average EEG magnitude (as defined in microvolts) within a frequency band over a specific time period (epoch).

Relative Magnitude: the relative EEG magnitude within a frequency band (absolute magnitude in a particular band divided by the total microvolts generated at a particular location in all bands)

Peak Amplitude: the peak amplitude of a frequency band during an epoch of time (defined in microvolts) Peak Frequency: the peak frequency within a band during an epoch of time (defined in_frequency)

Symmetry: the peak amplitude symmetry between two locations in a particular bandwidth—i.e. defined as (A−B)/(A+B), where A & B are electrode locations.

Peak Frequency: the peak frequency within a band during an epoch of time (defined in frequency)

Connectivity Measures

Coherence: the average similarity between the waveform morphology in a particular frequency band from two locations over an epoch (one-second period of time in this research). Conceptualized as the strength/number of connections between the two locations. Lexicor software provides an amplitude matching algorithm.

Phase: the time lag between waves from two locations in a particular band as defined by how soon after the beginning of an epoch a particular waveform at location #1 is matched in location #2 (amplitude)

Roland (1993) discusses issues of connectivity of the brain in terms of the anatomical organization of the neocortex, which contains six layers (with layer I being closest to the scalp, and approximately 3 mm thick). layer III send their axons to the other hemisphere or over longer distances intercortically. and phase figures The pyramidal cells (excitatory) in layer II and the upper part of layer III send their axons to the cortex in the same hemisphere while the). layer III send their axons to the other hemisphere or over longer distances intercortically. Apart from other subcortical considerations, these are the physiological foundations of coherence and phase_figures.

### FIG. 1—Nomenclature & Locations in the 10-20 system

### BRIEF SUMMARY

This patent addresses the relations between the quantitative EEG (QEEG) variables and the mental abilities of auditory and reading memory as well as problem solving ability, as assessed by the Ravens matrices. The QEEG variables examined were the “arousal” variables of magnitude, relative power, peak amplitude, & peak frequency while the connection variables involved Spectral Correlation Coefficients (SCC) and phase relations. The analysis presented in the figures document the significant positive and negative relations between the QEEG variables and performance on the cognitive task during the input and recall tasks (for auditory and reading memory) and during task performance on the Ravens matrices. The three claims made involved 1) the QEEG correlates of auditory memory (child and adult); 2) the QEEG correlates of reading memory (child and adult); 3) the QEEG correlates of problem solving (child and adult).

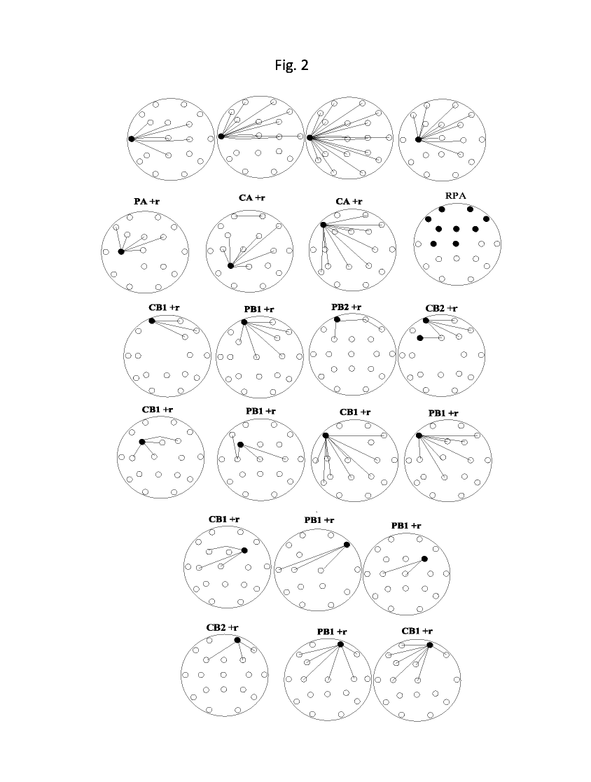

### FIG. 2—Adults listening to Stories (paragraphs) Positive Correlations between the QEEG variables and Memory score.

### RPA: Relative Power Alpha

### PT: Phase Theta CA: Coherence Alpha PA: Phase Alpha CB1: Coherence Beta1 (13-32 Hz)

### PB1: Phase Beta1 CB2: Coherence Beta2 (32-64 Hz) PB2: Phase Beta2

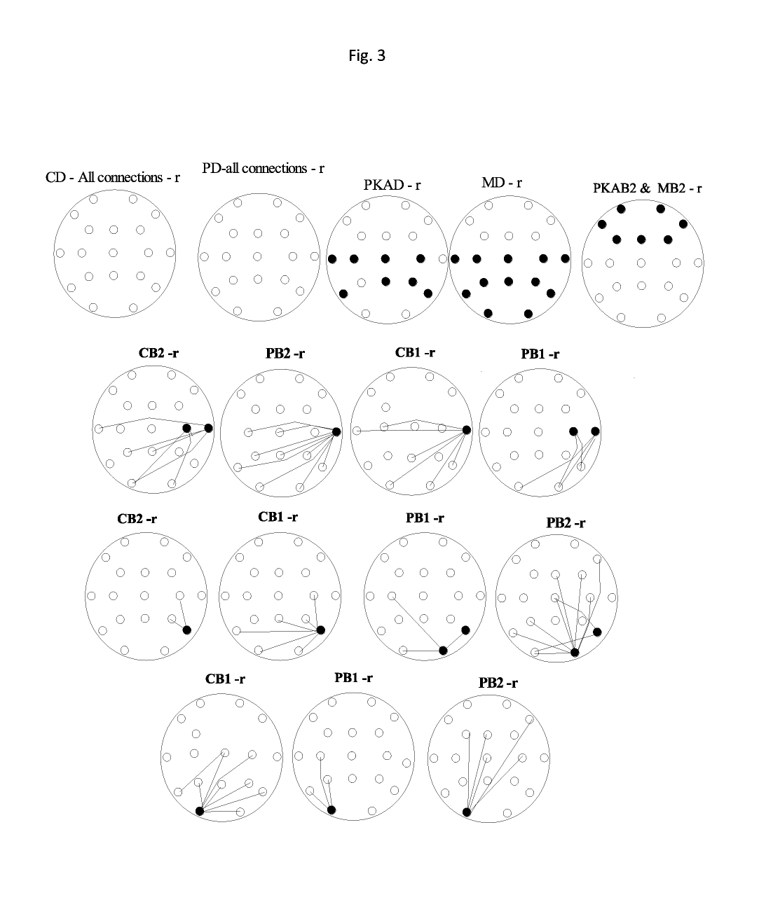

### FIG. 3—Adults Listening to Stories (paragraphs)

### Negative Correlations between QEEG variables and Memory Score

### PKAD: Peak Amplitude Delta MD: Magnitude Delta

### PKAB2: Peak Amplitude Beta2 MB2: Magnitude Beta2

### CD: Coherence Delta PD: Phase Delta CB1: Coherence Beta1 (13-32 Hz) PB1: Phase Beta1

### CB2: Coherence Beta2 (32-64 Hz) PB2: Phase Beta2

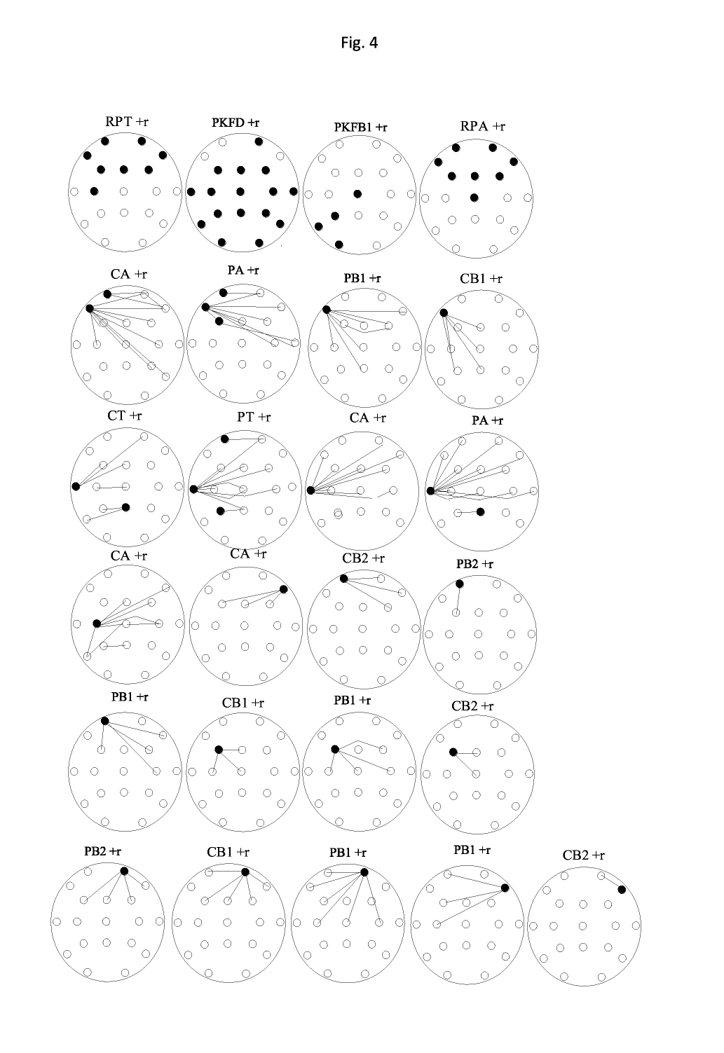

### FIG. 4—Adults Recalling Stories (paragraphs)

### Positive Correlations between QEEG variables and memory score

### RPT: Relative Power Theta RPA: Relative Power Alpha PKFD: Peak Frequency Delta

### PKFB1: Peak Frequency Beta1

### CT: Coherence Theta PT: Phase Theta CA: Coherence Alpha PA: Phase Alpha CB1: Coherence

### Beta1 (13-32 Hz) PB1: Phase Beta1 CB2: Coherence Beta2 (32-64 Hz) PB2: Phase Beta2

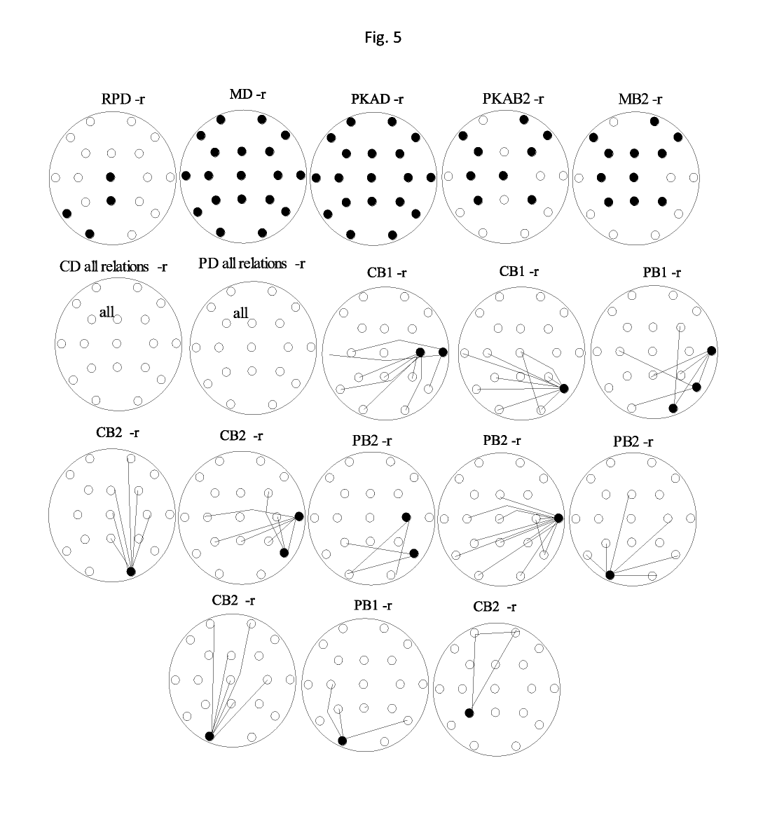

### FIG. 5—Adults Recalling Stories (paragraphs) quietly

### Negative Correlations between QEEG variables and memory score

### RPD: Relative Power Delta PKAD: Peak Amplitude Delta MD: Magnitude Delta

### PKAB2: Peak Amplitude Beta2 MB2: Magnitude Beta2

### CD: Coherence Delta PD: Phase Delta CB1: Coherence Beta1 (13-32 Hz)

### PB1: Phase Beta1 CB2: Coherence Beta2 (32-64 Hz) PB2: Phase Beta2

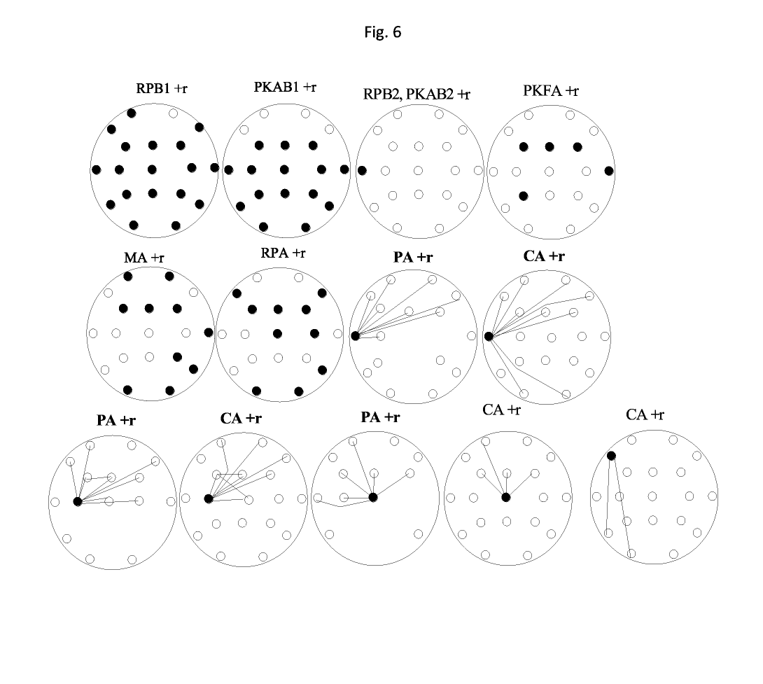

### FIG. 6—Children Listening to Stories (paragraphs) quietly

### Positive Correlations between QEEG variables and memory score

### RPA: Relative Power Alpha RPB1: Relative Power Beta1 RPB2: Relative Power Beta2

### PKAB1: Peak Amplitude Beta1 PKAB2: Peak Amplitude Beta2 PKFA: Peak Frequency Alpha

### CA: Coherence Alpha PA: Phase Alpha

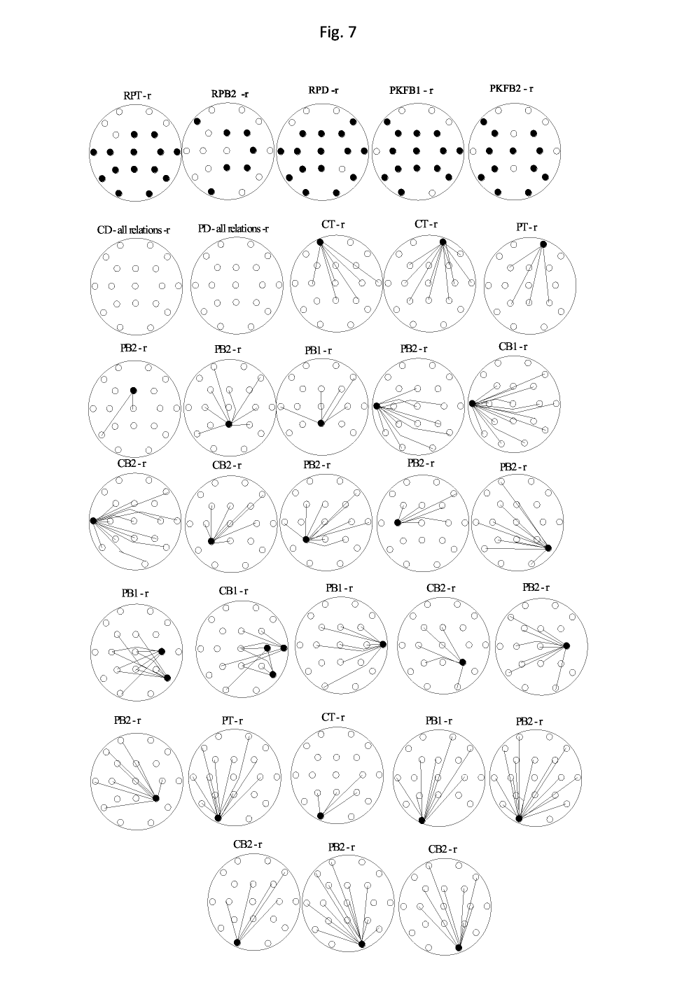

### FIG. 7—Children Listening to Stories (paragraphs) quietly

### Negative Correlations between QEEG variables and memory score

### RPD: Relative Power Delta RPT: Relative Power Theta RPB2: Relative Power Beta2

### PKFB1: Peak Frequency Beta1 PKFB2: Peak Frequency Beta2

### CD: Coherence Delta PD: Phase Delta CT: Coherence Theta PT: Phase Theta CB1: Coherence

### Beta1 (13-32 Hz) PB1: Phase Beta1 CB2: Coherence Beta2 (32-64 Hz) PB2: Phase Beta2

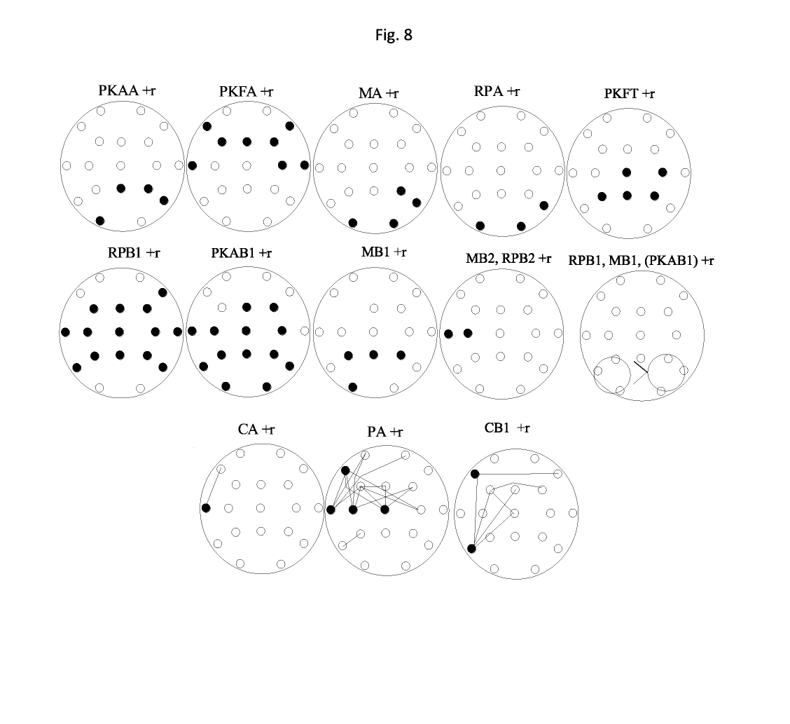

### FIG. 8—Child Recall Auditory quietly

### Positive Correlations between QEEG variables and memory score

### MA: Magnitude Alpha RPA: Relative Power Alpha

### CA: Coherence Alpha CB1: Coherence Beta1 (13-32 Hz) PB1: Phase Beta1

### CB2: Coherence Beta2 PB2: Phase Beta2

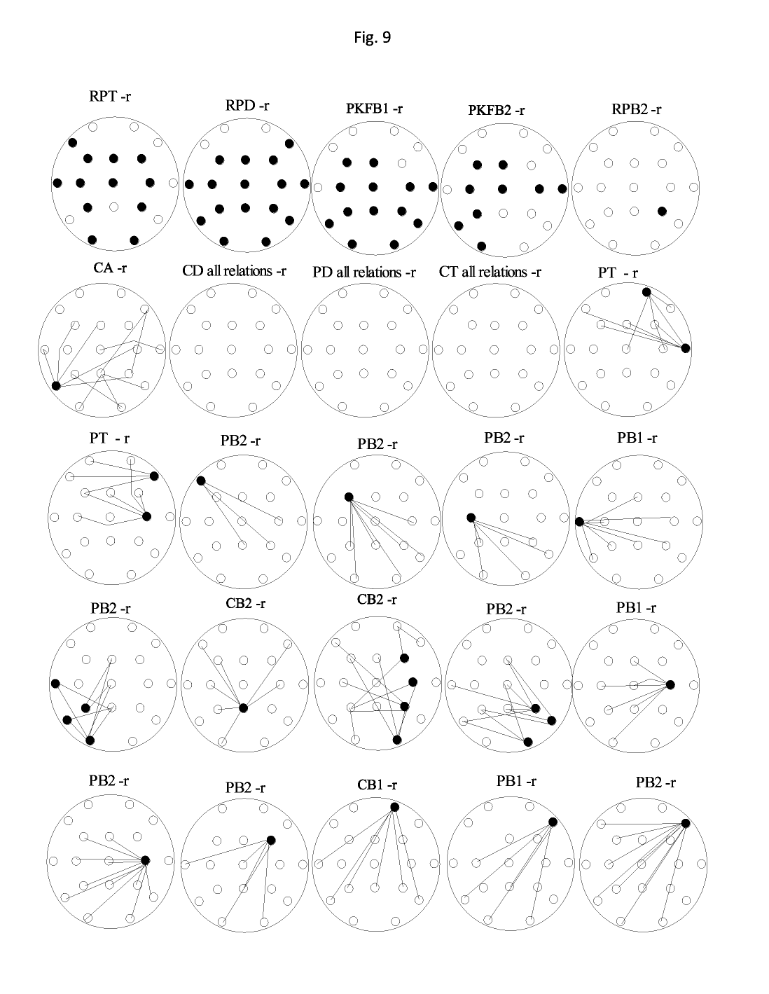

### FIG. 9—Child Recall Auditory quietly

### Negative Correlations between QEEG variables and memory score

### RPD: Relative Power Delta RPT: Relative Power Theta RPB2: Relative Power Beta2

### PKFB1: Peak Frequency Beta1 PKFB2: Peak Frequency Beta2

### CD: Coherence Delta PD: Phase Delta CT: Coherence Theta PT: Phase Theta CB 1: Coherence

### Beta1 (13-32 Hz) PB1: Phase Beta1 CB2: Coherence Beta2 (32-64 Hz) PB2: Phase Beta2

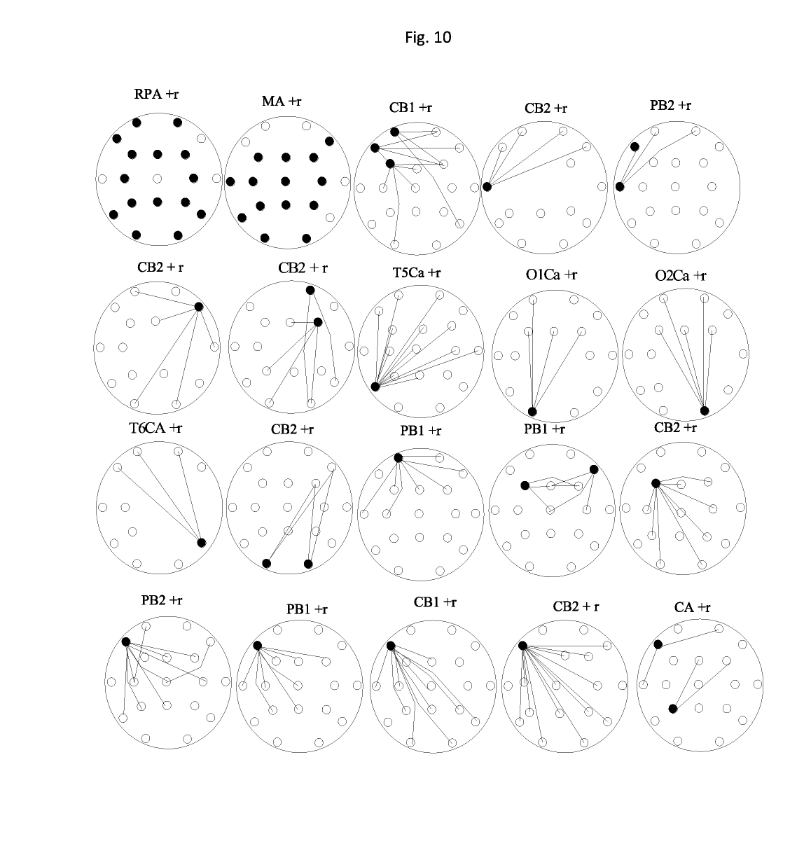

### FIG. 10—Adult Reading quietly

### Positive Correlations between QEEG variables and memory score

### RPA: Relative Power Alpha MA: Magnitude Alpha

### CA: Coherence Alpha CB1: Coherence Beta1 (13-32 Hz) PB1: Phase Beta1

### CB2: Coherence Beta2 (32-64 Hz) PB2: Phase Beta2

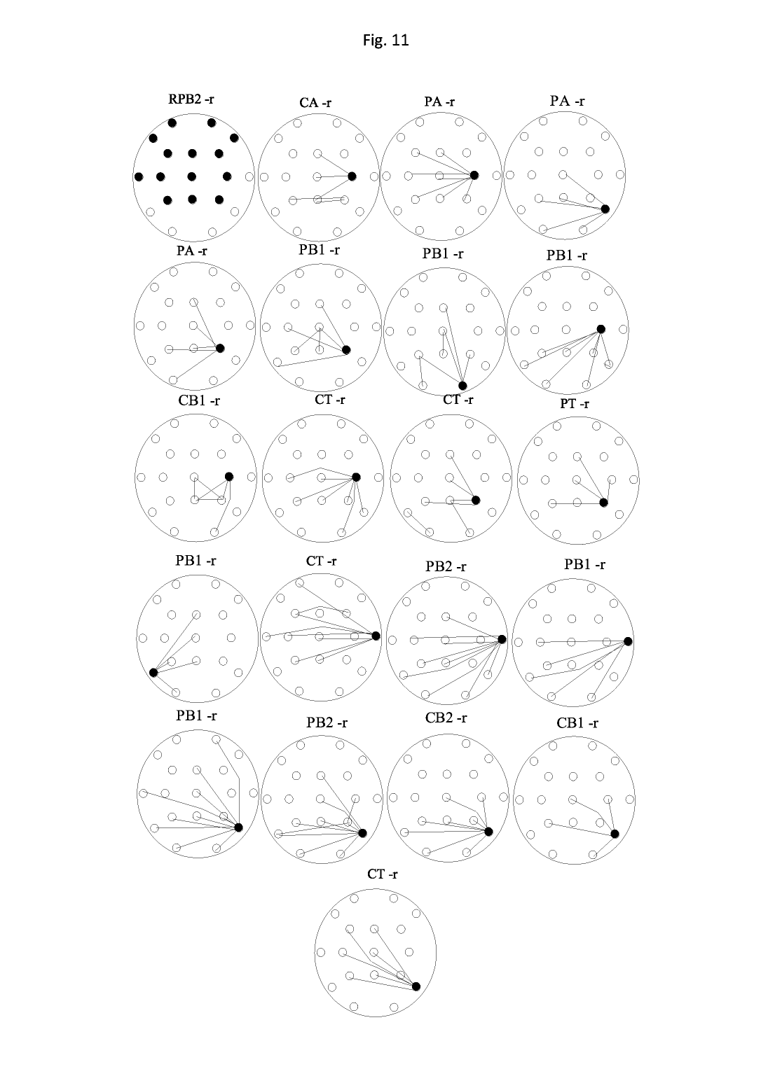

### FIG. 11—Adult Reading quietly

### Negative Correlations between QEEG variables and memory score

### RPB2: Relative Power Beta2

### CT: Coherence Theta PT: Phase Theta CA: Coherence Alpha PA: Phase Alpha CB1: Coherence

### Beta1 (13-32 Hz) PB1: Phase Beta1 CB2: Coherence Beta2 (32-64 Hz) PB2: Phase Beta2

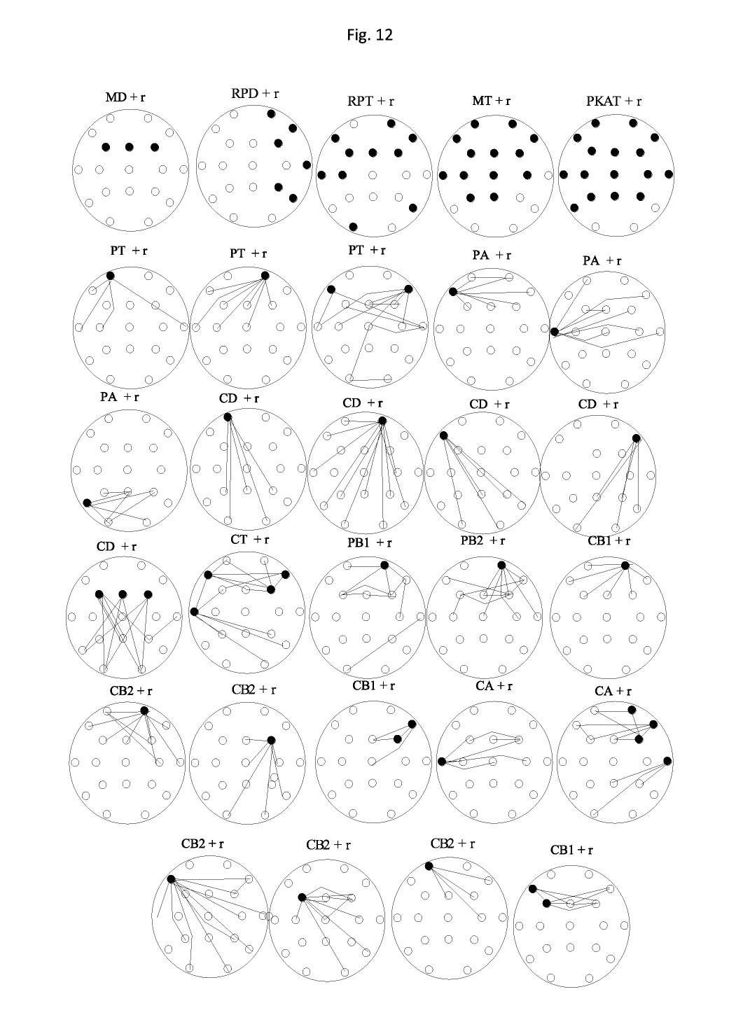

### FIG. 12—Adult Recall Reading quietly

### Positive Correlations between QEEG variables and memory score

### RPD: Relative Power Delta MD: Magnitude Delta RPT: Relative Power Theta

### MT: Magnitude Theta PKAT: Peak Amplitude Theta

### CD: Coherence Delta CT: Coherence Theta PT: Phase Theta CA: Coherence Alpha

### PA: Phase Alpha CB1: Coherence Beta1 (13-32 Hz) PB1: Phase Beta1 CB2: Coherence Beta2 (32-64 Hz) PB2: Phase Beta2

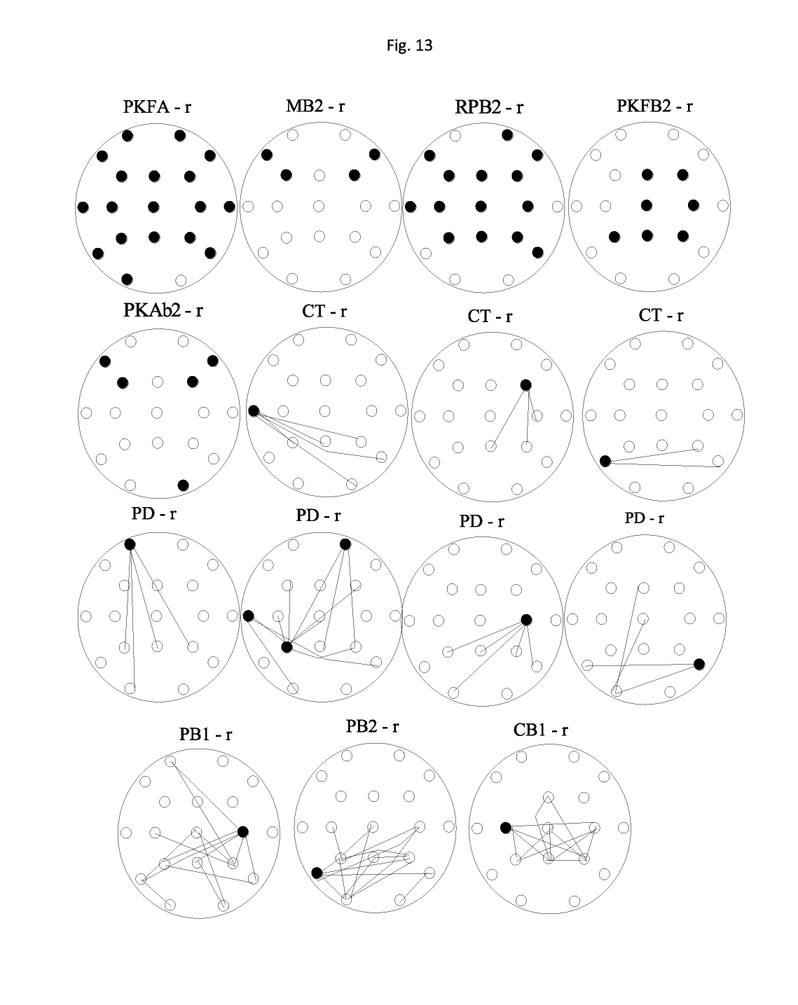

### FIG. 13—Adult Recall Reading quietly

### Negative Correlations between QEEG variables and memory score

### PKFA: Peak Frequency Alpha RPB2: Relative Power Beta2

### PKAB2: Peak Amplitude Beta2 MB2: Magnitude Beta2 PKFB2: Peak Frequency Beta2

### PD: Phase Delta CT: Coherence Theta CB1: Coherence Beta1 (13-32 Hz)

### PB1: Phase Beta1 PB2: Phase Beta2

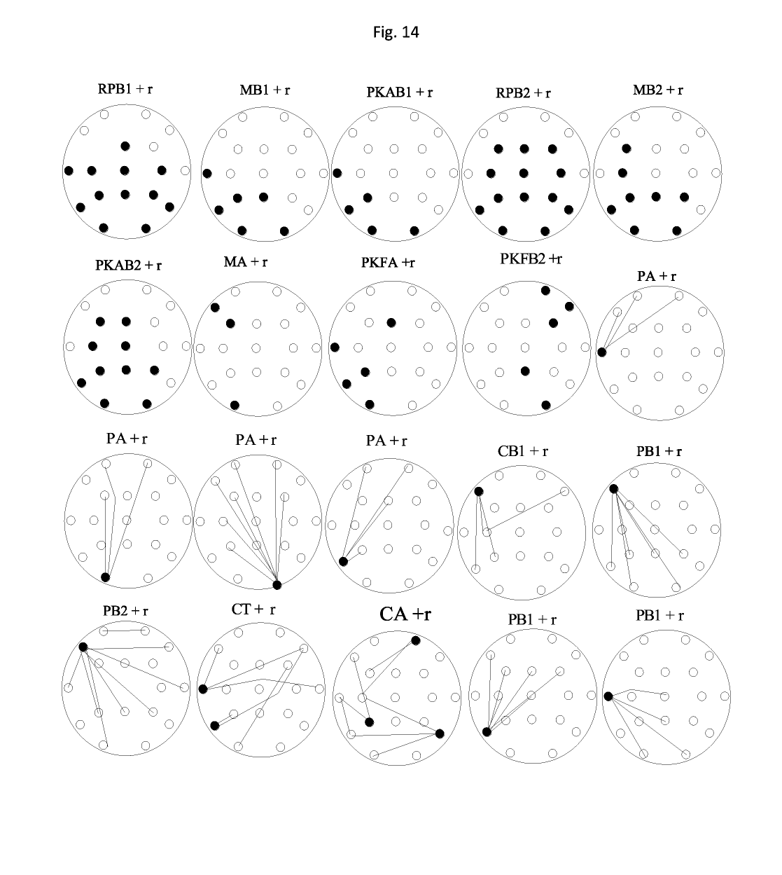

### FIG. 14—Child Reading quietly

### Positive Correlations between QEEG variables and memory score

### RPB1: Relative Power Beta1 RPB2: Relative Power Beta2 MB1: Magnitude Beta1

### MB2: Magnitude Beta2 PKAB1: Peak Amplitude Beta1 PKAB2: Peak Amplitude Beta2

### CA: Coherence Alpha CB1: Coherence Beta1 (13-32 Hz) CB2: Coherence Beta2

### PB1: Phase Beta1 PB2: Phase Beta2

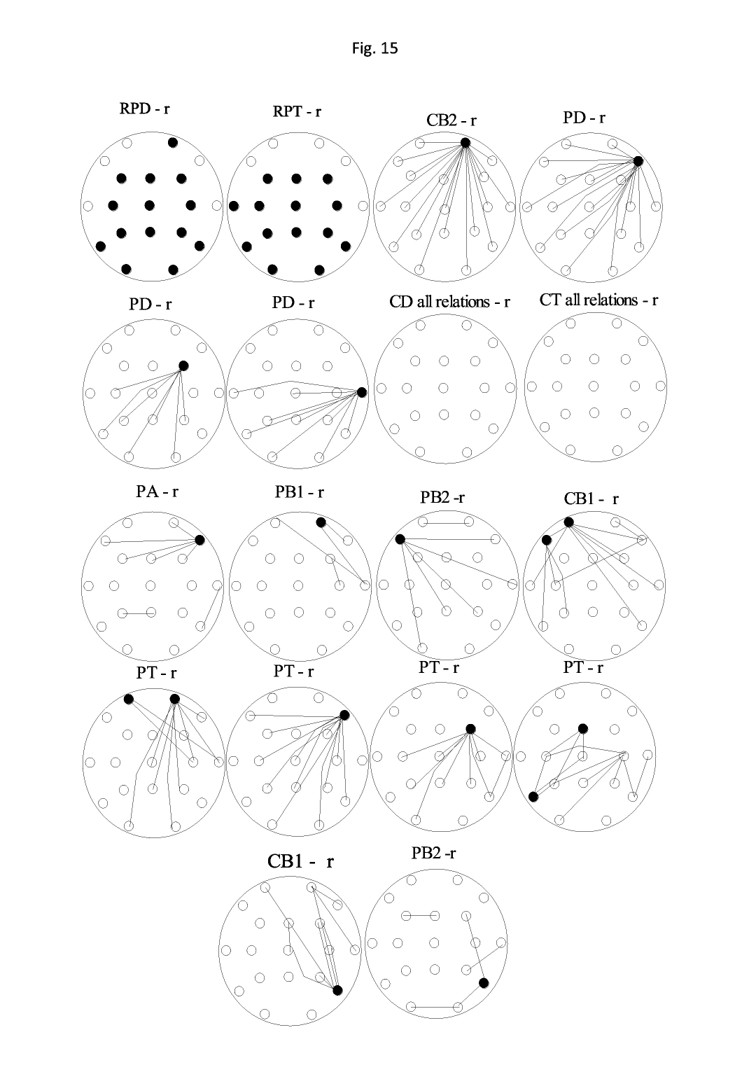

### FIG. 15—Child Reading quietly

### Negative Correlations between QEEG variables and memory score

### RPD: Relative Power Delta RPT: Relative Power Theta

### CD: Coherence Delta PD: Phase Delta PT: Phase Theta PA: Phase Alpha

### CB1: Coherence Beta1 (13-32 Hz) CB2: Coherence Beta2 (32-64 Hz) PB1: Phase Beta1

### PB2: Phase Beta2

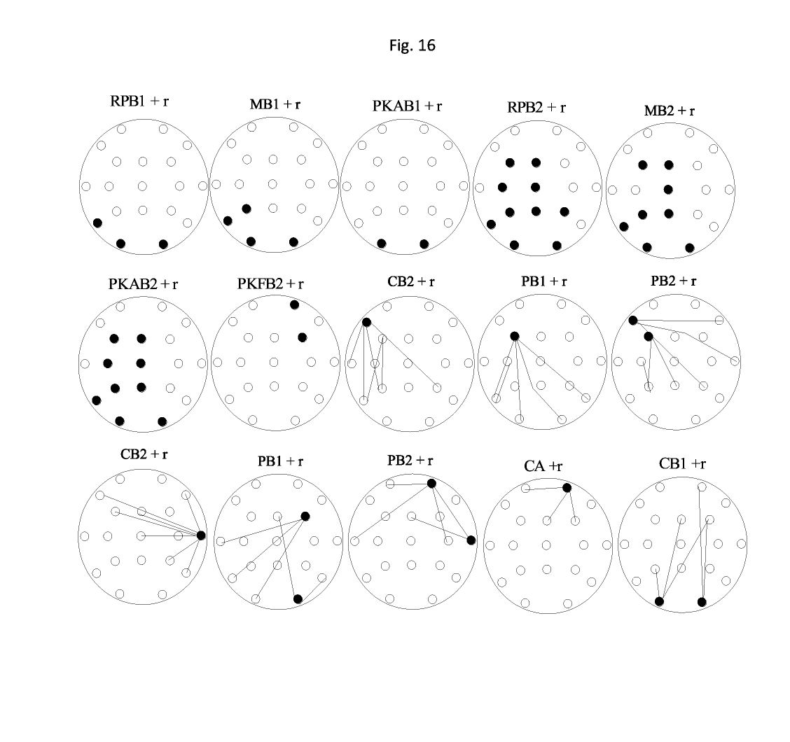

### FIG. 16—Child Recall Reading quietly

### Positive Correlations between QEEG variables and memory score

### RPB1: Relative Power Beta1 RPB2: Relative Power Beta2

### MB1: Magnitude Beta1 MB2: Magnitude Beta2 PKAB1: Peak Amplitude Beta1

### PKAB2: Peak Amplitude Beta2 PKFB2: Peak Frequency Beta2

### CA: Coherence Alpha CB1: Coherence Beta1 (13-32 Hz) PB1: Phase Beta1

### CB2: Coherence Beta2 (32-64 Hz) PB2: Phase Beta2

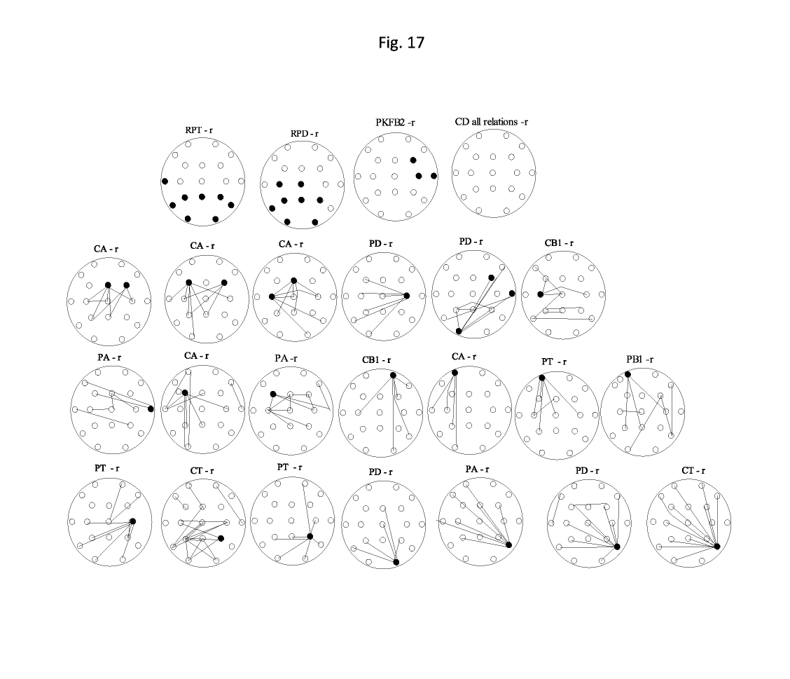

### FIG. 17—Child Recall Reading quietly

### Negative Correlations between QEEG variables and memory score

Insert FIG. 17

### RPD: Relative Power Delta RPT: Relative Power Theta PKFB2: Peak Frequency Beta2

### CD: Coherence Delta PD: Phase Delta CT: Coherence Theta PT: Phase Theta

### CA: Coherence Alpha PA: Phase Alpha CB1: Coherence Beta1 (13-32 Hz) PB1: Phase Beta1

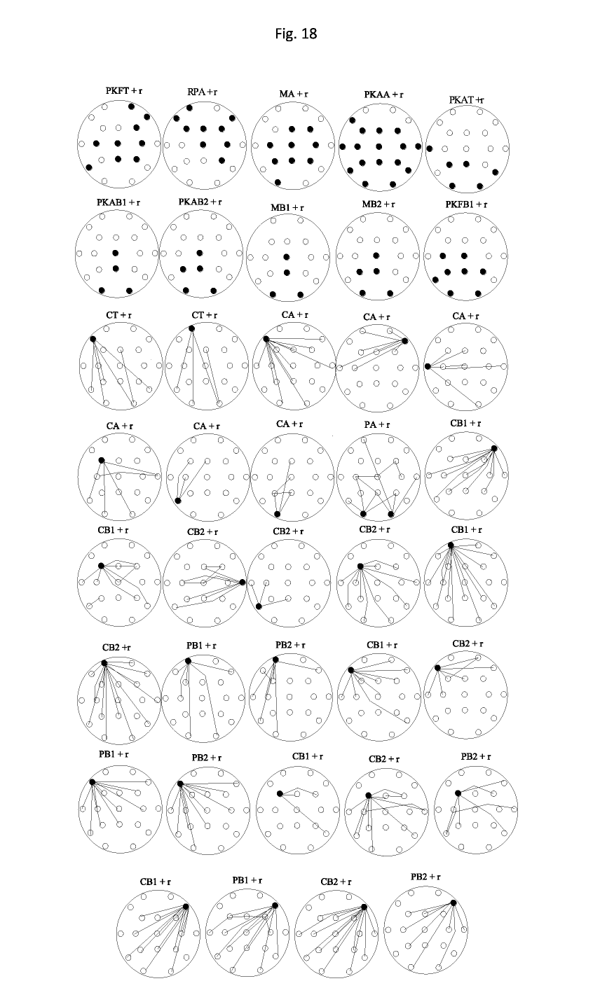

### FIG. 18—Adult Problem Solving—Ravens Matrices

### Positive Correlations between QEEG variables and Performance score

### RPA: Relative Power Alpha PKFT: Peak Frequency Theta PKFB1: Peak Frequency Beta1

### PKAT: Peak Amplitude Theta PKAA: Peak Amplitude Alpha PKAB1: Peak Amplitude Beta1

### PKAB2: Peak Amplitude Beta2 MA: Magnitude Alpha MB1: Magnitude Beta1

### MB2: Magnitude Beta2

### CT: Coherence Theta CA: Coherence Alpha PA: Phase Alpha CB1: Coherence Beta1 (13-32 Hz)

### PB1: Phase Beta1 CB2: Coherence Beta2 (32-64 Hz) PB2: Phase Beta2

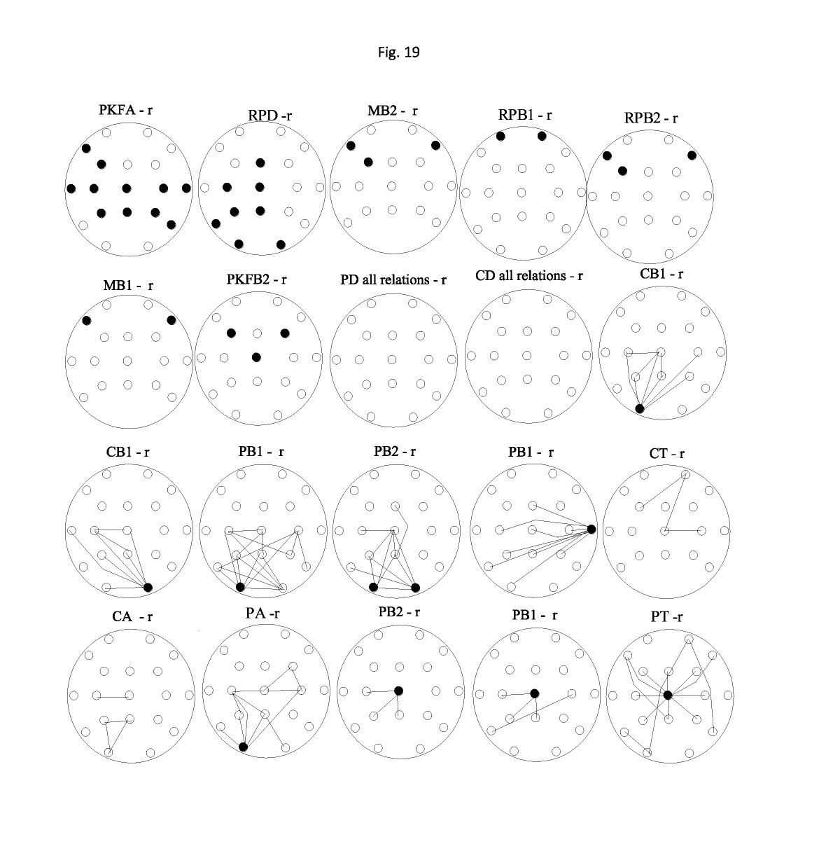

### FIG. 19—Adult Problem Solving—Ravens Matrices

### Negative Correlations between QEEG variables and Performance score

### RPD: Relative Power Delta RPB1: Relative Power Beta1 RPB2: Relative Power Beta2

### MB1: Magnitude Beta1 MB2: Magnitude Beta2 PKFB2: Peak Frequency Beta2

### PKFA: Peak Frequency Alpha PKFB2: Peak Frequency Beta2

### CD: Coherence Delta PD: Phase Delta CT: Coherence Theta PT: Phase Theta

### CA: Coherence Alpha PA: Phase Alpha CB1: Coherence Beta1 (13-32 Hz)

### PB1: Phase Beta1 PB2: Phase Beta2

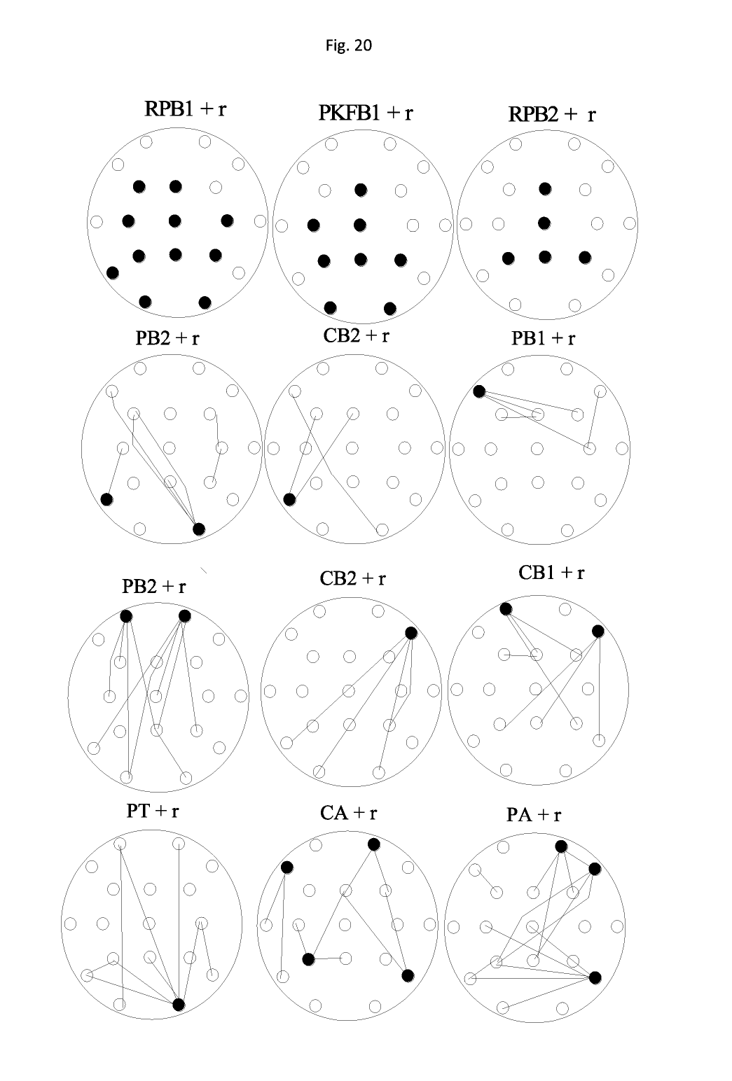

### FIG. 20—Child Problem Solving—Ravens Matrices

### Positive Correlations between QEEG variables and Performance score

### RPB1: Relative Power Beta1 RPB2: Relative Power Beta2 PKFB1: Peak Frequency Beta1

### PT: Phase Theta CA: Coherence Alpha PA: Phase Alpha CB1: Coherence Beta1 (13-32 Hz)

### PB1: Phase Beta1 CB2: Coherence Beta2 (32-64 Hz) PB2: Phase Beta2

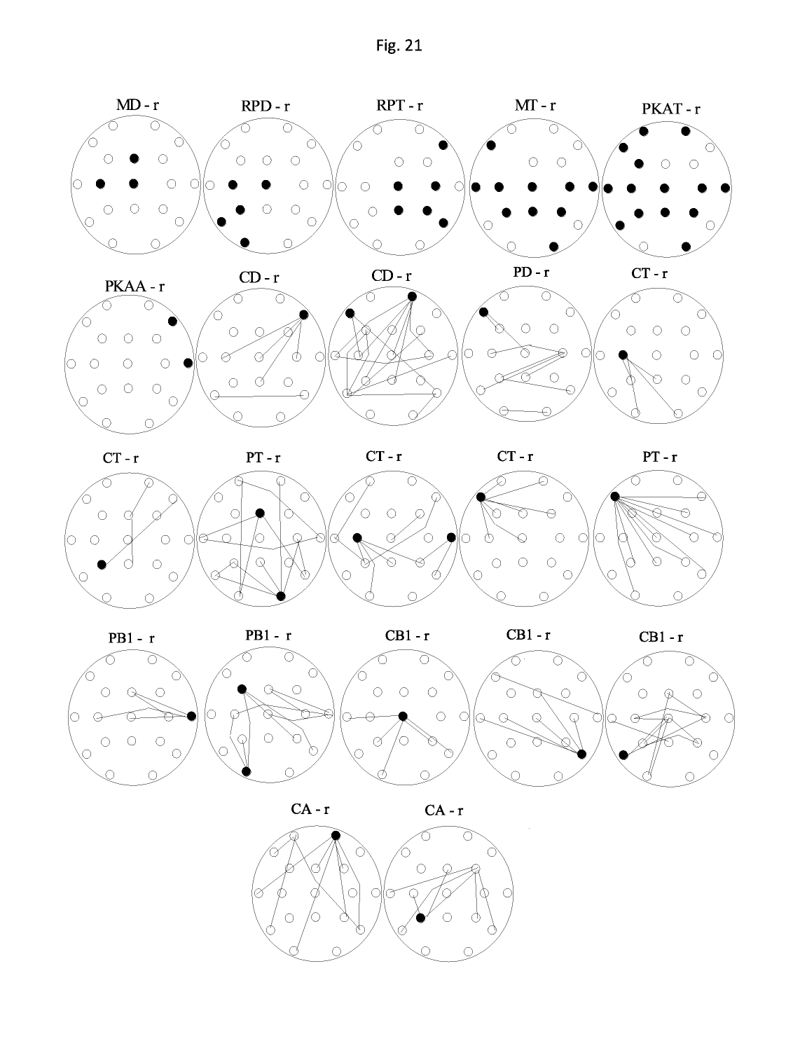

### FIG. 21—Child Problem Solving—Ravens Matrices

### Negative Correlations between QEEG variables and Performance score

### RPD: Relative Power Delta RPT: Relative Power Theta MD: Magnitude Delta MT: Magnitude

### Theta PKAT: Peak Amplitude Theta PKAA: Peak Amplitude Alpha

### CD: Coherence Delta PD: Phase Delta CT: Coherence Theta PT: Phase Theta

### CA: Coherence Alpha CB1: Coherence Beta1 (13-32 Hz) PB1: Phase Beta1

This patent makes the following 3 claims regarding the correlates between the QEEG variables and successful/unsuccessful cognitive performance. The verbal claims submitted in this section summarize the overall patterns while leaving more specific individual findings to the figures.

1.—Covers the QEEG positive and negative correlates of auditory memory during the input and immediate recall for adults and children. Successful auditory memory for adults (FIG. 2) is determined by the QEEG values of a) T3, C3, F7, P3 phase theta, phase alpha, & coherence alpha flashlight activity; b) within frontal lobe coherence and phase activity (beta1 and beta2) during the input stage.
The negatively related QEEG correlates of auditory memory in adults (FIG. 3) include delta relations (all coherence and phase values), frontal beta2 (peak amplitudes and magnitudes), posterior and central magnitude delta and peak amplitude delta values, right hemisphere (T4, C4, T6, O2) mini-flashlights in the beta1 and beta2 frequencies (coherence and phase) and O1 mini flashlights in the beta1 frequency (coherence and phase) and beta2 (phase) during the input stage.
The QEEG correlates of during successful quiet recall of auditory memory in adults (FIG. 4) are determined by the QEEG variables of frontal relative power of alpha and theta, diffusely located peak frequency of delta, left posterior peak frequency of beta1, mini flashlights from F7 (coherence and phase alpha & beta1), from T3 (coherence and phase theta and alpha), C3 (coherence alpha), Fp1 (coherence and phase beta2, phase beta1), F3 (coherence and phase beta1, coherence beta2), Fp2 (coherence and phase beta1, phase beta 2), F8 (coherence alpha, phase beta1, coherence beta2).
The QEEG correlates of unsuccessful quiet recall of auditory memory in adults (FIG. 5) are magnitude and peak amplitude of delta (all locations), relative power of delta (left posterior), peak amplitude and magnitude of beta2 (frontal to parietal locations), all coherence and phase delta relations, right hemisphere activity from C4, T4, T6, O2 in coherence and phase beta1 & beta2, O1 coherence and phase beta2 and phase beta1 as well as p3 (coherence beta2).
Successful auditory memory for children (FIG. 6) during the input stage is determined by the QEEG values of beta1 (relative power & peak amplitude—all locations), T3 (relative power & peak amplitude beta2), alpha (frontal peak frequency alpha, magnitude and relative power) and posterior alpha (magnitude, relative power), coherence and phase alpha at T3, C3, Cz and F7 (coherence alpha).
The negatively related QEEG correlates of auditory memory during the input stage in children (FIG. 7) are delta (coherence and phase—all locations), relative power of theta (posterior and central locations), relative power of delta (almost all locations), relative power of beta2 (right hemisphere and central locations), peak frequency beta1 & beta2 (almost all locations), coherence theta activity from frontal locations, phase beta1 and beta2 from Pz, T3 (coherence and phase beta2, coherence beta2), P3 (coherence and phase beta2), C3 (phase beta2), right hemisphere mini flashlights from T6, C4, T4, P4, O2 (coherence and phase beta1 and beta2), O1 (coherence and phase theta, phase beta1 and beta2, and coherence beta2).
The QEEG correlates of successful quiet recall of auditory memory in children (FIG. 8) are determined by the QEEG variables of alpha (posterior peak amplitudes, frontal peak frequencies, posterior magnitudes and relative power), peak frequency theta (parietal, central), beta1 (relative power & peak amplitudes—diffuse locations), magnitude beta1 (parietal, occipital locations), magnitude and relative power of beta2 at T3 and C3, coherence and phase alpha from T3, C3, Cz, F7) and T5 coherence beta2. The left posterior variables (beta1—relative power, magnitude and peak amplitude) are greater than the same variables in the right posterior locations.
The QEEG correlates of unsuccessful quiet recall of auditory material for children (FIG. 9) are relative power of theta and delta, peak frequency beta1 and beta2 (diffuse locations), posterior coherence alpha activity, all relations involving coherence and phase delta and coherence theta, right hemisphere mini flashlights from Fp2, T4, F8 (phase theta, left hemisphere phase beta2 mini flashlights (F7, F3, C3, T5, P3, O1), phase beta1 (T3), and coherence beta2 (Pz), right hemisphere mini flashlights from F4, C4, P4, O2, T6, Fp2, F8 (coherence and phase beta1 and beta2).

2. —Covers the QEEG successful and unsuccessful correlates of reading memory in the adult and child during the input and immediate recall periods. The QEEG correlates of successful reading memory during the input stage in adults (FIG. 10) are alpha (relative power, magnitudes—diffuse locations), frontal flashlights from Fp1, F7, F3, F8, Fp2, F4 (coherence and phase beta1 and beta2), T3 (coherence and phase beta2), coherence alpha from T5, O1, O2, T6, F7, P3 and coherence beta2 from O1 and O2.
The QEEG correlates of unsuccessful reading memory during the input stage in adults (FIG. 11) are relative power beta2 (diffuse locations), right hemisphere mini flashlights from C4, P4, T6, O2 (coherence theta, coherence and phase beta1 and beta2), and left hemisphere phase beta1 (T5).
The QEEG correlates of successful quiet recall of reading material in adults (FIG. 12) are delta (relative power—right hemisphere, frontal magnitude delta), theta (relative power—frontal locations; magnitude—diffuse locations), theta (peak amplitude—diffuse locations), frontal originating mini flashlights from Fp1, Fp2, F7, F8, F3, F4, Fz (phase and coherence theta, coherence delta, phase and coherence beta1 and beta2, coherence alpha, and T3 (coherence alpha and theta)
The QEEG correlates of unsuccessful quiet recall of reading material for adults (FIG. 13) are alpha (peak frequency—diffuse locations), beta2 (magnitudes—frontal locations; relative power—diffuse locations; peak frequency—parietal, central and right hemisphere locations, peak amplitudes (frontal), coherence theta (T3, F4, T5), phase delta (Fp1, Fp2, T3, P3, C4, T6), phase beta1 (C3, P4, T6, O2, T5, P3), phase beta2 (T5, O1, P4, C4), coherence beta1 (C3, C4, P4, Pz).
The QEEG correlates of successful reading memory during the input stage in children (FIG. 14) are beta1 (relative power—posterior and central locations; magnitudes—posterior and T3 locations; peak amplitudes—left posterior and T3), beta2 (diffuse locations; magnitudes—posterior and left hemisphere locations; peak amplitudes—diffuse locations, peak frequency (right frontal and right posterior locations), alpha (magnitude—left frontal; peak frequency—left posterior & T3), phase alpha (T3, O1, O2, T5), frontal coherence and phase beta1 and beta2, left hemisphere phase beta1 (T3 & T5), coherence theta and alpha (diffuse locations).
The QEEG correlates of unsuccessful reading memory during the input stage in children (FIG. 15) are delta and theta (relative power—diffuse locations), Fp2 (coherence beta2), phase delta (F8, F4, T4), coherence delta and theta (all relations), frontal mini flashlights from F8, Fp2, F7, Fp1, F4, Fz (phase theta, alpha, beta1, beta2; coherence beta1), right hemisphere mini flashlights from T6 (coherence beta1 and phase beta2).
The QEEG correlates of successful recall of reading memory during the recall stage in children (FIG. 16) are posterior beta1 (relative power (central), magnitude, peak amplitude), beta2 (relative power, magnitude, peak amplitudes—diffuse locations), peak frequency beta2 (right frontal), frontal mini flashlights from F7, F3, Fp2, F4 (phase beta1, coherence and phase beta1 and beta2, coherence alpha), occipital (O1, O2) mini flashlights in coherence and phase beta1, and T4 coherence and phase beta2.
The QEEG correlates of unsuccessful recall of reading memory during the recall stage in children (FIG. 17) are relative power of theta (diffuse locations), relative power of delta (diffuse locations), Fp2 (coherence beta2), right hemisphere phase delta (F8, F4, T4), coherence delta and theta (all relations), F8 (phase alpha), Fp2 (phase beta1), F7 (phase beta2), F7, F8 & Fp1 (coherence beta1), phase theta (Fp1, Fp2, F4, Fz, T5) and T6 (coherence beta1 and phase beta2).
The QEEG correlates of successful problem solving in adults (FIG. 18) are alpha (relative power—frontal locations; magnitude—central locations; peak amplitude—diffuse locations), theta (peak amplitude—occipital & Cz, Pz locations), beta1 (peak amplitude—occipital and Pz & Cz locations; magnitude occipital and Pz & Cz locations, beta2 (peak amplitudes—occipital and parietal locations; magnitude—occipital and parietal locations), peak frequency beta1 (posterior locations), coherence theta (Fp1, F7), coherence alpha (F7, f8, T3, F3, t5, O1), phase alpha (O1, O2), coherence beta1 (F8, F3, F4), coherence beta2 (T4, T5, F3), coherence and phase beta1 and beta2 (Fp1), coherence and phase beta1 and beta2 (F7), coherence beta1 & coherence beta2 and phase beta2 (F3) and F8 (coherence and phase beta1 and beta2).

3. covers the QEEG successful and unsuccessful problem solving in adults and children during the problem solving task. The QEEG correlates of unsuccessful problem solving in adults (FIG. 19) are alpha (peak frequency—diffuse locations), relative power of delta (diffuse locations), front beta activity (magnitude beta 2, relative power beta1 and beta2, magnitude beta1), phase and coherence delta activity (all relations), posterior coherence activity (O1, O2—coherence and phase beta1 and phase beta2) and T4 phase beta2, coherence theta and coherence and phase alpha (diffuse locations) and Cz (phase beta2, beta1, and theta)
The QEEG correlates of successful problem solving in children (FIG. 20) are beta1 (relative power—diffuse locations, peak frequency beta1—diffuse locations; relative power beta2—parietal and central locations), phase and coherence beta2 from T5, O2, and frontal mini flashlights from F7, Fp1, Fp2, F8 (coherence and phase beta1 & beta2), coherence alpha (F7, Fp2, P3, T6), phase alpha (Fp2, F8, T6), and phase theta (O2).
The QEEG correlates of unsuccessful problem solving in children (FIG. 21) are magnitude delta (Fz, Cz, C3), relative power delta (left posterior, C4, Cz), relative power of theta (right posterior, Cz, Pz, C4, F8), magnitude theta (central and parietal locations), peak amplitude theta (diffuse locations), peak amplitude alpha (F8, T4), coherence delta (F8, F7, Fp2), phase delta (F7, C4), coherence theta (C3, P3), phase theta (Fz, O2, F7), coherence theta (C3, T4, F7), phase beta1 (T3, F3, O1), coherence beta1 (Cz, T6, T5, O1), and coherence alpha (Fp1, Fp2, P3, F3).
The preceding 3 claims are substantially different from the 2001 patent claims regarding these specific cognitive abilities and represent a significant improvement in the knowledge in this area.
